SEQUENCE LISTING (12 sequences)

SEQ ID NO: 1 - protein, 831 aa, HUMAN
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 2 - protein, 57 aa, HUMAN
VLIVKKYVCGGRFLVHRYSVLQQHAEANGVDGVDALDTASHTNKSGYHDDSDEDLLE

SEQ ID NO: 3 - protein, 774 aa, Artificial Sequence
  acides amines 1-774 de la sortiline humaine
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAG

SEQ ID NO: 4 - protein, 70 aa, Artificial Sequence
  SEQ ID NO 2 liee par son extremite N-terminale de maniere covalente a la proteine Tat du VIH (SEQ ID NO 8)
GRKKRRQRRRPPQVLIVKKYVCGGRFLVHRYSVLQQHAEANGVDGVDALDTASHTNKSGYHDDSDEDLLE

SEQ ID NO: 5 - DNA, 171 bp, HUMAN
gtgctcattgtgaagaaatatgtctgtgggggaaggttcctggtgcatcgatactctgtgctgcagcagcatgcagaggccaatggtgtggatggtgtggatgctttggacacagcctcccacactaataaaagtggttatcatgatgactcagatgaggacctcttggaa

SEQ ID NO: 6 - DNA, 156 bp, Artificial Sequence
  SEQ ID NO 5 liee a SEQ ID NO 9
atgggtagaaagaaacgtagacaacgtagaaggcctccacaagtgctcattgtgaagaaagccaatggtgtggatggtgtggatgctttggacacagcctcccacactaataaaagtggttatcatgatgactcagatgaggacctcttggaatag

SEQ ID NO: 7 - protein, 84 aa, Artificial Sequence
  SEQ ID NO 2 liee par son extremite N-terminale de maniere covalente a SEQ ID NO 8 et pas on extremite C-terminale a SEQ ID NO 1O
GRKKRRQRRRPPQVLIVKKYVCGGRFLVHRYSVLQQHAEANGVDGVDALDTASHTNKSGYHDDSDEDLLEGKPIPNPLLGLDST

SEQ ID NO: 8 - protein, 12 aa, Adenovirus type 1
GRKKRRQRRRPQ

SEQ ID NO: 9 - DNA, 42 bp, Adenovirus type 1
atgggtagaaagaaacgtagacaacgtagaaggcctccacaa

SEQ ID NO: 10 - protein, 14 aa, Artificial Sequence
  Etiquette synthetique V5
GKPIPNPLLGLDST

SEQ ID NO: 11 - protein, 18 aa, Artificial Sequence
  model ampiphatic peptide
KLALKLALKALKAALKLA

SEQ ID NO: 12 - protein, 19 aa, Artificial Sequence
  DPV1047
VKRGLKLRHVRPRVTRMDV

(19) United States
(12) Patent Application Publication
(10) Pub. No.: US 2023/0212251 A1
(43) Pub. Date: Jul. 6, 2023

(54) PEPTIDES DERIVED FROM SORTILIN

(71) Applicant: UNIVERSITE DE LIMOGES, Limoges (FR)

(72) Inventors: Camille GRANET, Bonnac-la-Côte (FR); Eric LAPEYRONNIE, Saint Front La Riviere (FR); Thomas NAVES, Ambazac (FR); Paul-François GALLET, Limoges (FR); Marie-Odile JAUBERTEAU, Limoges (FR)

(21) Appl. No.: 17/634,425

(22) PCT Filed: Aug. 10, 2020
(86) PCT No.: PCT/EP2020/072431

§ 371 Date: Feb. 10, 2022

(30) Foreign Application Priority Data
Aug. 13, 2019 (FR) 1909182

(51) Int. Cl. C07K 14/705

(52) U.S. Cl. CPC C07K 14/705; C07K 2319/10; C07K 2319/40; A61K 38/00

(57) ABSTRACT

An isolated polypeptide derived from human sortilin, including an amino acid sequence which has at least 80% identity with the sequence SEQ ID NO 2 Val Leu Ile Val Lys Lys Tyr Val Cys Gly Gly Arg Phe Leu Val Mis Arg Tyr Ser Val Leu Gin Gin Mis Ala Glu Ala Asn Gly Val Asp Gly Val Asp Ala Leu Asp Thr Ala Ser Mis Thr Asn Lys Ser Gly Tyr Mis Asp Asp Ser Asp Glu Asp Leu Leu Glu on condition that the polypeptide does not contain the sequence SEQ ID NO 3 or a sequence having at least 80% identity with the aforementioned sequence SEQ ID NO 3, for use as a drug.

### REFERENCE TO AN ELECTRONIC SEQUENCE LISTING

The contents of the electronic sequence listing (1B30271.txt; Size: 18164 bytes was created on Aug. 29, 2022) is herein incorporated by reference in its entirety.

### BACKGROUND

The present invention relates to peptides derived from sortilin and use thereof in a pathology associated with sortilin deregulation, such as cancers, in particular non-small cell lung cancers, neurological disorders, in particular Parkinson's disease, Alzheimer's disease, coronary diseases and atherosclerosis.

Sortilin (NCBI, NP-002950, 18 Jun. 2019) or NTSR3 (Neurotensin Receptor 3) was initially discovered and characterized in the nervous system. Its gene SORT1 (NM 002959) is located on the short arm of chromosome 1 at locus 1p13.21 (109 309 568-109 397 951). Unlike the first two neurotensin receptors described, NTSR1 and NTSR2 (respectively on chromosomes 20 and 2; NM_002531 and NM_012344), which are G-protein-coupled receptors with seven transmembrane domains, sortilin is a type-I receptor. Its extracellular portion shares a VPS10 (vacuolar protein sorting) domain with four other members; SORCS1 (sortilin-related VPS10 domain containing receptor 1), SORCS2 (sortilin-related VPS10 domain containing receptor 2), SORCS3 (sortilin-related VPS10 domain containing receptor 3) and SORLA (sortilin-related receptor 1). The set of members of this VPS10 family functions both as sorting receptor in the Golgi compartments and transporter of soluble vacuolar proteins.

Sortilin expression is ubiquitous, but remains higher in the brain, spinal cord, testicles and skeletal musculature. It is synthesized in the endoplasmic reticulum (ER) in the form of a precursor protein for 831 amino acids (aa) and corresponds to sequence SEQ ID NO 1. In its N-terminal portion, a signal peptide making it possible to route the sortilin through the ER and the Golgi network precedes a propeptide of 44 aa. This propeptide masks the domain VPS10, thus preventing, from the ER, binding of the ligands of sortilin in its immature form. In the Golgi, a furin convertase cleaves the propeptide, thus liberating it for access to the VPS10 domain and activating the sorting functions of the sortilin. The liberated propeptide, which corresponds to the residues of amino acids 1 to 44, is biologically active. A molecule derived from this propeptide (residues 17 to 28), modified by the addition of the APLRP residues, was generated and named “spadin”. The latter has antidepressant effects by inhibition of the TREK-1 potassium channel (US2012322060 and EP 3099313). Once mature, sortilin binds different ligands via its VPS10 domain, such as the precursors of nerve growth factor (pro-NGF) or brain-derived neurotrophic factor (pro-BDNF), progranulin, lipoprotein lipase, type 4 glucose transporter (GLUT4), RAP12 and including its own propeptide. Sortilin thus would appear to play a regulatory role on the quantity of neurotrophic factors to be secreted by directing the excess to the lysosome (PMID: 21730062 Evans S. F. et al. J. Biol. Chem. (2011), 286(34), 29556-29567).

At the level of the cell membrane, sortilin can i) either by recycled to the endosomes or the trans-Golgi via motifs contained in its intracellular portion, or ii) ensure its second function as co-receptor via its VPS10 domain and form heterodimers with membrane receptors, such as the death domain receptor p75NTR or to the neurotrophin receptors (TrkA, —B, -C) promoting the activation of cell signalling. The VPS10 domain constitutes the whole of the luminal/extracellular domain of the sortilin in the form of a cysteine-rich structure preserved between yeast and human. Its crystalline structure (PDB: 3F6K, https://www.rcsb.org/) was obtained at neutral pH in the presence of its natural ligand, neurotensin. The VPS10 domain is divided into three domains; the N-terminal portion (sortilin residues 45-576) forms a helical structure formed of ten sub-structures organized in 13 sheets at the centre of which the C-terminal portion of the neurotensin binds, followed by two small domains called 10CCa and 10CCb (sortilin residues 577-633 and 634-716, respectively). These two domains constitute the 10CC module containing few secondary structures and strongly interacting with the helical structure. In its intracellular sorting functions, sortilin is exposed to pH variations between the different subcellular compartments, in particular in the late endosomes, where the acidity of these latter allows the release of the ligands associated with their receptors. The crystalline structure of the VPS10 domain at acid pH (5.5) reveals global distortion of the helical structure followed by homodimerization of the sortilin then blocking access to the ligands.

The intracellular portion of sortilin contains a sequence homology with the C-terminal domain of the mannose-6-phosphate (M6PR) receptors, in particular that which is independent of the cations or cation-independent mannose-6-phosphate receptor (CI-M6PR). In fact, the C-terminal sequences of the CI-M6PR and of the sortilin share eight common amino acids (DD SDEDLL). This octapeptide comprises two functional sites; the casein kinase II phosphorylation site (serine residue) and a dileucine motif (LL). These two sites represent the essential functional motifs of the M6PRs for their traffic between the Golgi compartments and the late endosomes. Although the casein kinase II phosphorylation site is essential for recruitment of the adaptor protein 1 (AP-1) necessary for the formation of the trans-Golgi vesicles, the dileucine motif is involved in the subsequent sorting mechanisms. Mutations generated within the octapeptide change the interaction of the CI-M6PR with the VHS domain of the Golgi-associated, gamma adaptin ear containing, ADP ribosylation factor binding proteins (GGAs). Owing to the fact that the GGAs bind the clathrin necessary for specific transport, these mutations block the exit of the CI-M6PRs from the Golgi. In addition, these same mutations modify the sorting of the CI-M6PRs from the endocytic recycling compartments (ERC) to the other intracellular organelles during their retrograde transport, without thereby modifying their rates of internalization starting from the cell membrane. As a result, this motif represents an active sorting/internalization signal that directs the receptors at different levels of their intracellular traffic. Besides the dileucine motif (residues 829 and 830 of the protein), sortilin contains at least two other sorting signals, tetrapeptide YSVL and the FLVHRY motif (respectively in positions 792-795 and 787-792 of sortilin) immediately adjacent to the transmembrane domain. The tetrapeptide YSVL abides by a consensus motif YXXZ, where Z represents a bulky or aromatic hydrophobic residue. This sequence is involved in the internalization and rapid sorting of membrane proteins such as LRP (lipoprotein receptor-related protein), the membrane proteins of the trans-Golgi network 38/41 (TGN 38/41) and the 46 kDa M6PR 20-22. The FLVHRY sequence constitutes another sorting/internalization signal according to the motif (F/Y) XXXX (F/Y) necessary for the traffic of the M6PR receptors and those of the LRP family.

Sortilin, in particular human sortilin, is known for its different roles in Alzheimer's disease, Parkinson's disease and diabetes. It is also involved in the effects of neurotensin on the growth of cancer cells of prostate, pancreatic or colonic origin (Dal Farra C. et at. Int J Cancer, 2001; 92, 503-9) and has important functions in the control of lipoprotein metabolism (Current Opinion in Lipidology, 2011, 22(2), 79-85). Numerous studies based on artificial knockout of sortilin, which makes it possible to identify the roles of the different domains of which it is composed, have allowed the demonstration of a domain interacting with the amyloid precursor protein (APP). (Miao Yang et al., PLOS ONE, 8, No 5, 21 May 2013 Page e63049, XP055671832).

Bronchial adenocarcinomas represent the greatest proportion (˜80%) of the non-small cell lung cancers (NSCLC), which remain the principal cause of death from cancer in France and worldwide. The NSCLCs belong to the cancers that are the most difficult to treat with limited progression-free survival in comparison with the cancers of the breast and colon, which share common clinical characteristics. Given this observation, intense efforts have been deployed to improve knowledge of the mechanisms facilitating the initiation and progression of NSCLCs. Accordingly, molecular studies reveal that the latter have the highest rate of mutation load on the tyrosine kinase receptor (RTK) activity, such as the epidermic growth factor receptor (EGFR), which remains the archetype of the RTKs. Somatic mutations in its tyrosine kinase (TK) domain generate a constitutively active form of the receptor, independently of its ligand fixation, and aberrantly activate the signalling pathways associated with cell proliferation. Accordingly, “cell addiction” is initiated, placing the mutations that activate EGFR as principal oncogenic factors.

Surprisingly, there is no difference between the frequency or the type of mutation between early-stage or advanced NSCLCs. Thus, from a therapeutic point of view, these mutations offer the opportunity to precisely target EGFR and to orient the clinical strategies towards therapies known as “personalized”, such as tyrosine kinase activity inhibitors (TKIs). TKIs limit both the intensity and the duration of the proliferative signals of the EGFR, thus reducing the aggressivity of the tumours and the progression of the disease in patients. Including TKIs in clinical protocols increases patients' overall survival; however, it still remains less than 15% at 5 years. In fact, the clinical advantages of TKIs inevitably drop, independently of the stage of the disease. The main reason is that a secondary mutation takes place in EGFR exon 20 (T790M), inhibiting fixing of the inhibitors, thus reactivating its kinase activity and tumour progression. However, a crucial question remains with respect to the appearance or the pre-existence of this mutation known as “acquired resistance” mutation.

It appears that EGFR has functions other than the transduction of a signal at the cell surface, in particular a role of transcription factor (in the cell nucleus), which would induce the expression of mitogenic genes that are resistant to treatment. The EGFR transcription program would promote TKI resistance. Thus, the EGFR receptor, independently of its kinase activity, would promote treatment escape.

In earlier studies (Al-Akhrass H. et al., Nat. Commun. (2017); 8(1): 1182), the inventors demonstrated a new role for sortilin, a protein belonging to the VPS10 family, on the regulation and stabilization of EGFR. They showed that sortilin actively intervenes in internalization and degradation of EGFR. Sortilin inhibition leads to the accumulation of EGFR in a hyperactive state, forcing the tumour cells to proliferate and survive.

Histopathological and molecular studies conducted by the inventors on a cohort of 72 patients suffering from a bronchial adenocarcinoma revealed increasing repression of sortilin with the pathological degrees of the disease. In fact, sortilin expression is intimately linked to better patient survival. Its expression remains high in tumours that are weakly active (slow-multiplying) and differentiated. In other words, sortilin expression indicates a good prognosis in adenocarcinomas, particularly in the case of tumours having high EGFR expression (Al-Akhrass H. Doctoral thesis “A new role of sortilin in the control of EGFR retrograde trafficking to limit tumour growth” https://www.theses.fr/204476984).

The inventors have also shown that in tumours having a mutation sensitizing EGFR to TKIs, sortilin is highly expressed. Conversely, in tumours having the mutation T790M, insensitizing EGFR to TKIs, sortilin expression is weak. Overexpression of sortilin in a cell model expressing the mutated EGFR T790M, reverses the resistant phenotype of these cells and “re-sensitizes” these latter to TKIs (Al-Akhrass H. Doctoral thesis cited above).

The inventors have also shown that EGFR and sortilin mutually interact through their extracellular portion, i.e. the VPS10 domain of sortilin, with the extracellular portion of EGFR. Expression of a truncated form of sortilin (devoid of its intracellular portion, i.e. of amino acids 778-831) disrupts the internalization of EGFR, suggesting that the intracellular portion of sortilin would act to route EGFT to the degradation compartments. Surprisingly, this portion can be released within the cells following cleavage by gamma-secretases, and more precisely by presenilin 1 (PSEN1). This cell mechanism is not specific to sortilin and remains common to cleavage of the intracellular portion of known proteins such as Notch and APP. The peptides relating to these two proteins are biologically active and have nuclear functions. The inventors' research studies have made it possible to characterize and to isolate, starting from sortilin, a polypeptide having an intracellular biological activity. Analyses carried out directly on tumour fragments from patients have shown that expression of EGFR is inversely correlated with that of said polypeptide named SICD (sortilin intracellular domain) and suggest that the SICD polypeptide may play a role in the degradation and/or expression of EGFR.

Thus, there is a need to have available new compounds for the treatment of pathological states associated with sortilin deregulation.

There is also a need to have available tools for the prognosis of pathological states involving sortilin and in particular a change in its expression or even its biological activity.

The aim of the present invention is to meet these needs.

### SUMMARY

The inventors detected this peptide, naturally generated in the cells following proteolytic cleavage of sortilin, isolated and modified it in order to allow its penetration into the cells and targeting of the nucleus, and demonstrated the antineoplastic effect thereof.

According to a first aspect, a subject of the invention is an isolated sortilin-derived polypeptide corresponding to its C-terminal portion, for use thereof as a medicament. A further subject of the invention is an isolated sortilin-derived polypeptide corresponding to its C-terminal portion linked with a cell-penetrating peptide and the nucleotide sequence coding for the polypeptide according to the invention, expression vectors comprising said nucleotide sequence and host cells transformed by said expression vectors.

According to a second aspect, a subject of the invention is a pharmaceutical composition comprising said polypeptide or the corresponding nucleotide sequence.

According to a third aspect, a subject of the invention is a medicament containing the polypeptide or the corresponding nucleotide sequence or the pharmaceutical composition, for use thereof in the treatment of a disease associated with sortilin deregulation.

Finally, according to a fourth aspect, a subject of the invention is the use of the polypeptide as a label or as a prognostic agent of at least one pathology associated with sortilin deregulation, in particular cancers, (in particular non-small cell lung cancers), neurological disorders, in particular Parkinson's disease and Alzheimer's disease, coronary diseases and atherosclerosis.

A subject of the present invention is an isolated sortilin-derived polypeptide comprising or consisting of an amino acid sequence having at least 80% identity with sequence SEQ ID NO 2, provided that said polypeptide does not contain sequence SEQ ID NO 3 or a sequence having at least 80% identity with said sequence SEQ ID NO 3 or one of its pharmaceutically acceptable salts for use thereof as a medicament.

According to the invention, one or more amino acids of the polypeptide can be replaced by a non-natural amino acid or a natural or non-natural amino acid analogue, provided that the three-dimensional structure of the polypeptide is respected. Apart from the 20 natural amino acids (Ala, Arg, Asn, Asp, Cys, Gln, Glu, Gly, His, Ile, Leu, Lys, Met, Phe, Pro, Ser, Thr, Trp, Tyr and Val), there are others, natural or not, which are described for example by Hunt S. (The Non-Protein Amino Acids: in Chemistry and Biochemistry of the Amino Acids, Barrett, Chapman and Hall, 1985). Thus, an aromatic amino acid such as phenylalanine can be replaced by 3,4-dihydroxy-L-phenylalanine, 3-iodo-L-tyrosine, triiodothyronine, L-thyroxine, phenylglycine (Phg) or nor-tyrosine (norTyr). Phg and norTyr and other amino acids, including Phe and Tyr, can be substituted by, for example, a halogen, a —CH3, —OH, —CH2NH3, —C(O)H, —CH2CH3, —CN, —CH2CH2CH3, —SH group or another group. Any amino acid can be substituted by the D-form of the amino acid.

With respect to the non-natural amino acids or the analogues of natural and non-natural amino acids, a certain number of substitutions are possible, alone or in combination. A person skilled in the art will be able to make such substitutions in light of their general knowledge. Thus, by way of example, glutamine residues (Glu) can be substituted by gamma-hydroxy-Glu or gamma-carboxy-Glu. Tyrosine residues can be substituted by an alpha-substituted amino acid such as L-alpha-methyl phenylalanine or by analogues such as: 3-amino-tyr; tyr(CH3); tyr(P03(CH3)2); tyr(SO3H); beta-cyclohexyl-ala; beta-(1-cyclopentenyl)-ala; beta-cyclopentyl-ala; beta-cyclopropyl-ala; beta-quinolyl-ala; beta-(2-thiazolyl)-ala; beta-(triazole-1-yl)-ala; beta-(2-pyridyl)-ala; beta-(3-pyridyl)-ala; amino-phe; fluoro-phe; cyclohexyl-gly; tBu-gly; beta-(3-benzothienyl)-ala; beta-(2-thienyl)-ala; 5-methyl-trp and α-methyl-trp. Proline residues can be substituted by homopro (L-pipecolic acid); hydroxy-pro; 3,4-dehydro-pro; 4-fluoro-pro; or alpha-methyl-pro; or a cyclized N(alpha)-C(alpha) amino acid analogue with the structure: n=0, 1, 2, 3. Alanine residues can be substituted by an alpha-substituted or N-methylated amino acid such as alpha-amino isobutyric acid (aib), L/D-alpha-ethylalanine (L/D-isovaline), L/D-methylvaline or L/D-alpha-methylleucine or a non-natural amino acid such as beta-fluoro-ala. Alanine can also be substituted by: n=0, 1, 2, 3. The glycine residues can be substituted by alpha-aminoisobutyric acid (aib) or L/D-alpha-ethylalanine (L/D-isovaline).

In some cases, an amino acid can be replaced with a non-essential amino acid of natural origin, such as taurine.

The term “sortilin” denotes a polypeptide comprising 831 amino acids and corresponding to sequence SEQ ID NO 1 or a polypeptide having at least 80% identity with said sequence SEQ ID NO 1.

According to the invention, the polypeptides are derived from sortilin of human or animal origin. They can be of natural or recombinant origin. Finally, they can be synthetic.

The term “isolated polypeptide” denotes a polypeptide obtained by a method known to a person skilled in the art, and in particular by the recombinant route or by chemical synthesis, said polypeptide then being separated from its initial environment by partial or total purification. Similarly, the term “isolated nucleic acid” denotes a nucleic acid obtained by a method known to a person skilled in the art, and in particular according to any known method of genetic engineering or by chemical synthesis, followed by purification of said nucleic acid. An isolated polypeptide according to the invention thus differs from a natural polypeptide but is not limited to a particular method of preparation or synthesis. Similarly, an isolated nucleic acid according to the invention differs from a natural amino acid but is not limited to a particular method of synthesis.

Preferably, a polypeptide according to the invention is produced by the recombinant route, according to techniques well known to a person skilled in the art, specialized in this field. Production of a recombinant protein includes in particular selecting the host intended for production, for example a bacterium, a eucaryotic cell from yeast or mammal or from a transgenic animal, and the design of a synthetic nucleotide sequence suitable for said production, in particular by selecting a production vector and selecting the codons used in order to optimize production. A recombinant protein can be produced in the form of a fusion protein, which combines the protein of interest with a “tag” domain intended to allow the identification and/or the purification of said protein of interest. Purification of the protein of interest is then carried out according to a method from among the numerous protocols available to a person skilled in the art.

In the expression “has at least 80% identity with said sequence” the percentage is purely statistical and the differences between the two nucleotide sequences or between amino acids can be randomly distributed over their whole length. The percentage sequence identity between two sequences is defined after alignment of the sequences to be compared. When a position in one of the sequences is occupied by the same base or the same amino acid, the molecules are identical at this position. A degree of identity between two sequences is a function of the number of identical positions between the two sequences. The comparisons of sequences can be carried out by using any algorithm intended for this purpose known to a person skilled in the art.

According to a particular aspect of a polypeptide according to the invention, the amino acid sequence has at least 80%, preferably at least 85%, at least 90%, 91%, 92%, 93%, 94%, 95%, 96%, 97%, 98%, at least 99% or 100% identity with sequence SEQ ID NO 2.

When the polypeptide according to the invention corresponds to SEQ ID NO 2, then it corresponds to the amino acids 775-831 of human sortilin. It belongs to the intracellular domain of sortilin and is named SICD (sortilin intracellular domain).

According to another particular aspect of the invention, the polypeptide corresponding to SEQ ID NO 2 is covalently bound to a cell-penetrating peptide. This covalent bond can be formed either at the N-terminal end, or at the C-terminal end of the peptide.

According to the invention, by cell-penetrating peptide (CPP) is meant molecules that can translocate into the cells without damaging the membranes. There may be mentioned by way of example the CPPs quoted by Borelli A. et al. (Molecules 2018, 23, 295) and those quoted by Guidotti G. et al. (Trends in Pharmacological Sciences, April 2017, 38(4)), in particular the HIV1 Tat protein, penetratin, peptide originating from the homeodomain of the Antennapedia protein, p28 originating from azurin, VP22 from the herpes simplex virus (HSV-1) or even synthetic peptides such as MAP (model amphipathic peptide) of sequence SEQ ID NO 11 or DPV1047 of sequence SEQ ID NO 12 described by Guidotti G. et al. (Trends in Pharmacological Sciences, April 2017, Vol. 38, No. 4). These peptides can pass through the cell membrane and reach the cytoplasm and/or the nucleus of the cells. They can therefore transport a wide variety of biologically active molecules into the cell. Moreover, some of these CPPs have their own activity capable of reinforcing that of the molecule that they transport.

In an advantageous embodiment of the invention, the cell-penetrating peptide is the Tat protein and the polypeptide according to the invention has sequence SEQ ID NO 4 corresponding to the polypeptide of sequence SEQ ID NO 2 covalently bound to the Tat protein of sequence SEQ ID NO 8. The presence of the HIV1 Tat protein offers the opportunity to pass through the blood-brain barrier and the bronchial and intestinal mucous membranes. Accordingly, the active range of the peptide can be extended to locally advanced forms, to deep metastases such as cerebral metastases.

A subject of the invention is also an isolated nucleotide sequence selected from the following polynucleotides:

a. a polynucleotide coding for the polypeptide according to the invention of sequence SEQ ID NO 2 and consisting of sequence SEQ ID NO 5,

b. a polynucleotide that has at least 80% or more, at least 85% or more, at least 90% or more, at least 95% or more homology with the polynucleotide of sequence SEQ ID NO 5 and

c. a polynucleotide that is degenerate with respect to polynucleotides a and b. The genetic code is degenerate (or redundant), which means that there can be several codons to denote one and the same amino acid. Thus, a degenerate polynucleotide is a polynucleotide having a different sequence, but suitable for carrying out the same function or producing the same result as the initial polynucleotide that corresponds to sequence SEQ ID NO 2. The degenerate polynucleotide will have a sequence different to that of polynucleotides a and b, but corresponds to the rules of genetic code degeneracy, which are accessible via the following link:

https://people.bath.ac.uk/jm2219/biology/degenerate.htm.

In an advantageous embodiment of the invention, the polynucleotide corresponding to SEQ ID NO 5 is covalently bound to a polynucleotide coding for a cell-penetrating peptide. When the polynucleotide codes for the HIV1 Tat protein, then the polynucleotide has sequence SEQ ID NO 6.

A further subject of the invention is an expression vector comprising a polynucleotide coding for a polypeptide according to the invention.

According to the invention “vector” denotes a nucleic acid molecule capable of transporting a nucleotide sequence to which it is bound, or of allowing the expression of the protein coded by a nucleotide sequence to which it is bound operationally (expression vector).

In an advantageous embodiment of the invention, the nucleotide vector comprises sequence SEQ ID NO 5 or sequence SEQ ID NO 6.

A further subject of the invention is a host cell comprising a polynucleotide coding for a polypeptide according to the invention or a vector according to the invention. The host cell can be any cell commonly used and is in particular selected from the group comprising bacteria, yeasts, human cell lines, animal cells.

A further subject of the invention is a pharmaceutical composition comprising a polypeptide or one of the pharmaceutically acceptable salts thereof, or a polynucleotide according to the invention or one of the pharmaceutically acceptable salts thereof in combination with a pharmaceutically acceptable excipient. This composition can also contain another active agent.

According to the invention, by “pharmaceutically acceptable excipient” is meant any substance other than the active substance, intended to contribute a consistency, a taste, a colour to a medicament, while avoiding any interaction with the active principle. The pharmaceutically acceptable excipient according to the invention will be selected according to the pharmaceutical form and the desired mode of administration, from the usual excipients that are known to a person skilled in the art, in light of their administration to humans or animals.

By “active agents” is meant any substance other than the polypeptide or polynucleotide according to the invention and having a therapeutic effect. There may be mentioned in particular by way of example the conventional chemotherapeutic molecules (such as cisplatin) or pharmacological receptor tyrosine kinase inhibitor (RTKI) molecules, or signalling routes used in targeted treatment or receptor-inhibiting antibodies or immune suppression routes (checkpoint inhibitors of T-lymphocytes) such as gefitinib, or even erlotinib. The present list being non-limitative.

According to the invention, the pharmaceutical compositions can be presented in any pharmaceutical form suitable for local, regional, systemic or continuous administration. There may be mentioned by way of example the following administration routes: oral, sublingual, subcutaneous, parenteral, intramuscular, intravenous, topical, local, intratracheal, intranasal, ocular, intraperitoneal routes, via endoparietal, transdermal or rectal diffusion, or via any other administration route. The appropriate forms of administration comprise forms via oral route such as tablets, soft or hard capsules, powders, granules and oral solutions or suspensions, sublingual, buccal, intratracheal, intraocular, intranasal forms of administration, via inhalation; the topical, transdermal, subcutaneous, intramuscular or intravenous forms of administration, the rectal forms of administration and implants. For topical application, it is possible to use the compounds according to the invention in creams, oil-in-water or water-in-oil emulsions, gels, ointments, patches, solutions or lotions.

A further subject of the invention is a polypeptide or a polynucleotide or a pharmaceutical composition according to the invention as a medicament. According to the invention, they can be used as is, or in the form of a pharmaceutically acceptable salt. There may be mentioned by way of example, acid addition salts with an organic or inorganic acid such as acetic acid, succinic acid and hydrochloric acid, the ammonium salts or alkali metal salts such as sodium or potassium salt.

In an advantageous embodiment of the invention, the polypeptide according to the invention or the pharmaceutical composition according to the invention can be used as a medicament in combination with at least one second antineoplastic treatment, said second antineoplastic treatment being administered before, after, or at the same time as the polypeptide or the polynucleotide or the pharmaceutical composition.

By way of example of second antineoplastic treatment, there may be mentioned surgery, chemotherapy (treatment with a molecule having antineoplastic activity), radiotherapy (administration of a radiotherapy agent), hormone therapy (administration of hormone derivatives), toxin therapy, immunotherapy and cryotherapy.

A polypeptide or one of the pharmaceutically acceptable salts thereof according to the invention or a polynucleotide or one of the salts thereof according to the invention or a pharmaceutical composition according to the invention can be used in a pathology associated with sortilin deregulation, such as for example cancers, in particular non-small cell lung cancers, neurological disorders, in particular Parkinson's disease, Alzheimer's disease, coronary diseases and atherosclerosis.

By “sortilin deregulation” is meant variations in the quantity of sortilin.

A further subject of the invention is a method for the treatment of pathologies associated with sortilin deregulation such as for example cancers, in particular non-small cell lung cancers, neurological disorders, in particular Parkinson's disease, Alzheimer's disease, coronary diseases and atherosclerosis, comprising the administration of a polypeptide or a polynucleotide or one of the salts thereof according to the invention to a patient in need of them.

A polypeptide according to the invention comprising or consisting of an amino acid sequence having at least 80% identity with sequence SEQ ID NO 2

Val Leu Ile Val Lys Lys Tyr Val Cys Gly Gly Arg Phe Leu Val His Arg Tyr Ser Val Leu Gln Gln His Ala Glu Ala Asn Gly Val Asp Gly Val Asp Ala Leu Asp Thr Ala Ser His Thr Asn Lys Ser Gly Tyr His Asp Asp Ser Asp Glu Asp Leu Leu Glu

provided that said polypeptide does not contain sequence SEQ ID NO 3, or a sequence having at least 80% identity with said sequence SEQ ID NO 3 whether or not it is covalently bound to an intracellular penetrating peptide can also be used as a label or as a prognostic agent for pathologies associated with sortilin deregulation, such as for example cancers, in particular non-small cell lung cancers, neurological disorders, in particular Parkinson's disease and Alzheimer's disease, coronary diseases and atherosclerosis.

Thus a subject of the invention is a method for in vitro or ex vivo prognosis of a pathological state associated with sortilin deregulation, such as for example cancers, in particular non-small cell lung cancers, neurological disorders, in particular Parkinson's disease and Alzheimer's disease, coronary diseases and artherosclerosis, comprising a first assessment of the expression of the polypeptide of sequence SEQ ID NO 2 present in a biological sample obtained from said patient, comparing said first assessment with assessments of the expression of said polypeptide determined in a population of healthy subjects, where a variation of the expression of said polypeptide in said biological sample with respect to the expression determined for healthy subjects is indicative of the present of the pathological state.

According to the invention, detecting the polypeptide can be done by any technique known to a person skilled in the art; it can in particular be done directly in the pathological tissues of the patients, such as the tumours or biopsies or blood samples. This detection can be done by the western blot technique, the technique combining an exclusion chromatography coupled with mass spectrometry, the competitive ELISA technique, using specific antibodies coupled or not with an enzyme or isotope tracer in order to detect the polypeptide by imaging.

### BRIEF DESCRIPTION OF THE DRAWINGS

The following FIGS. 1 to 4 and Examples 1 to 3 illustrate the invention:

FIG. 1.A represents the detection by western blot of the synthetic peptide of sequence SEQ ID NO 7 according to the invention in the intracellular medium according to Example 1. “SICD peptide” corresponds to the increasing concentrations of peptide of SEQ ID NO 7 (SICD peptide coupled with V5 tag and Tat) used according to Example 1. “IB: anti-V5” quantity of intracellular peptide measured with anti-V5 antibody; “IB: anti-SORT-ICD” quantity of intracellular peptide measured with the antibody directed against the intracellular portion of sortilin; “IB: anti-actin” internal control.

FIG. 1.B represents the visualization of this synthetic peptide at the nucleus level via immunofluorescence according to Example 1. DAPI labels the cell nuclei, while the V5 tag makes it possible to visualize the intracellular location of the synthesized peptide. In fact, when two-dimensional images are taken and the two labels superimposed, the distribution of the V5, and thus that of the synthesized peptide, is superimposed on that of the nucleus. This distribution is punctiform, in the form of spots located at the nucleus level. Three-dimensional imaging makes it possible to assess the distribution of these spots in the depth of the nuclei, along the z axis, and not only on their surface as 2D imaging allows. Thus, as shown in FIG. 1.B, the synthetic peptide forms spots which are distributed within the cell nuclei.

FIG. 2 represents the cell survival assessment, measured by XTT (Cell Proliferation Kit II-Roche), in the presence of the SICD peptide coupled with the V5 tag and the Tat and corresponding to sequence SEQ ID NO 7 at different concentrations after 24, 48 and 72 hours' incubation according to Example 2.

FIG. 3 represents the real-time quantification of the fixing of annexin V induced by the pro-apoptotic effect of the SICD peptide coupled with the V5 tag and the Tat corresponding to sequence SEQ ID NO 7 according to Example 3.

FIG. 4 represents the real-time quantification of the activation of the caspase 3 and 7 induced by the pro-apoptotic effect of the SICD peptide coupled with the V5 tag and the Tat corresponding to sequence SEQ ID NO 7 according to Example 3.

### DETAILED DESCRIPTION

1. Cells and Cell Culture

The embryonic kidney line HEK293T (ATCC® CCL-1573™) and the bronchial adenocarcinoma cell lines A549 (ATCC® ^{CCL185™}) and H1975 (ATCC® CCL-5908™) originate from the American Type Culture Collection (ATCC). The line H3255 was kindly donated by Madame Sylvie Gazzeri (DR2 INSERM, U823, Institut Albert Bonniot, Grenoble). The cells are cultured in a DMEM (Dulbecco's Modified Eagle Medium) culture medium+Glutamax (ref: 10566016, Gibco, ThermoFisher Scientific) made up with 10% fetal calf serum (ref: 9215-50, H2B), 1% pyruvate (ref: 11360070, Gibco, ThermoFisher Scientific) and 1% penicillin/streptomycin (ref: 10378016, Gibco, ThermoFisher Scientific). The DMEM+Glutamax culture medium thus made up will be known as “complete medium” in the present application. The cells are kept in a humid atmosphere in an incubator at 37° C. containing 5% CO2 (Binder, ThermoFisher Scientific). For subculturing and weekly maintenance of the cells, the culture medium containing the dead cells and debris is removed, the cell layer is rinsed with PBS 1×(phosphate buffer saline) (ref: 14190144, Gibco, ThermoFisher) then the cells are detached from their support by addition of trypsin-EDTA (0.05%) (ref: 25300054, Gibco, ThermoFisher) for 5 minutes in a humid atmosphere in an incubator at 37° C. containing 5% CO2. Once detached, the cells are collected in a 15-or 50-mL tube (respectively ref: 62554502; 62547254, Sarstedt) and counted under the microscope (×40) on a counting chamber of the “Malassez” type (ref: 631-0975; VWR). In parallel, the cell viability is estimated on the counting chamber after colouring a sample of the cell suspension quarter-diluted in a solution of trypan blue 0.4% (ref: 15250061, Gibco, ThermoFisher). The cells are then centrifuged at 100 x-g at 20° C. (ref: 75004380, Sorvall, ThermoFisher) for 10 minutes. The cell pellet thus obtained is either returned to suspension so that the cells are at the appropriate density for the various experiments planned, or stored dry at −80° C. for subsequent use. A concentration of 10.10³ cells/cm² is usually used for the seeding of each cell subtype used in this invention. The cells are seeded in culture flasks of 25 or 75 cm² (respectively ref: 83.3910.002 and 83.39.11.502, Sarstedt), or even in culture plates with 6 or 24 wells (respectively ref: 83.3920 and 83.3922, Sarstedt) depending on the experiments to be carried out.

2. Long-Term Cell Storage: Freezing/Defrosting

The cultured cells are detached, collected and centrifuged as previously described. They are then taken up in suspension in 1 mL fetal calf serum (FCS) made up with 10% of a cryoprotector, dimethyl sulfoxide (DMSO) (ref: D-8418, Sigma-Aldrich) at a rate of 3×10⁶ cells per cryotube (ref: 72380002, Sarstedt). These are placed in a freezing container (ref: 479-0966, VWR) containing isopropanol (ref: 563935, Sigma-Aldrich) for 24 hours to 1 week for progressive freezing, before being stored in a container of liquid nitrogen at −196° C. The defrosting stage must be rapid in order to obtain optimal cell viability. Then, immediately it is removed from the liquid nitrogen, the cryotube is placed in a water bath at 37° C. until complete defrosting of the cell suspension. The cells are taken up in 10 mL of complete medium and centrifuged (300 x-g, 10 minutes at 25° C.) in order to eliminate the DMSO. They are then returned to culture according to the conditions previously described.

3. Extraction of the proteins under denaturing conditions Extraction of the whole proteins is carried out directly on the cell layers, after eliminating the supernatants and rinsing the cells in PBS 1×, or even on cell pellets previously stored at −80° C. It is carried out by incubating the cells with the Laemmli lysis buffer (62.5 mM Tris-HCl ref: 11814273001 ROCHE, pH 6.8, 2% SDS ref: L-4509, Sigma-Aldrich, 25% glycerol ref: G5516, Sigma-Aldrich) made up with 1% (v/v) protease inhibitor cocktail (ref: P8340-1ML, Sigma-Aldrich) and 1% (v/v) phosphatase inhibitor cocktail (ref: P0044-1ML, Sigma-Aldrich), in the proportions 100 pL of buffer for 1.10⁶ cells for 30 min over ice. In the case of extraction on the cell layer, a scraper (ref: 179693PK, Nunc, ThermoFisher) is used to collect the cells in the lysis buffer. Lysis is then completed by ultrasound parametered at 60 Hz, amplitude 2 s, for 1 minute (ref: SONIVCX-130-220, VWR). The lysates are then centrifuged at 17,000×g (ref: 75002442, Sorvall, ThermoFisher) for 20 min at 4° C., the supernatant containing the proteins is transferred to a sterile 1.5 mL tube (ref: 0030120086, Eppendorf), and the proteins are assayed by the Bradford method.

4. Protein Assay

The protein concentration of the various samples is assessed by the Bradford method (Bradford 1976). The test is conducted by incubating the diluted samples (or the different concentrations of the bovine albumin standard (ref: 5000206, Bio-Rad)) with the reagents from the kit DC™ Protein Assay Kit II (ref: 5000112, Bio-Rad) following the manufacturer's protocol. The absorbance reading is carried out at 595 nm on a spectrophotometer (ref: 51119000, ThermoFisher). The protein concentration of each sample is estimated with respect to the straight line obtained with the BSA concentration range (0; 125; 250; 500; 750; 1000; 1500 and 2000 μg/mL). Once the concentrations of the samples have been determined by this method, the necessary volume of proteins is sampled, then equilibrated as a function of each sample, generally 20 pL. These latter are made up respectively with 0.01% of bromophenol blue and 5% of 3-mercaptoethanol before being denatured at 95° C. for 5 minutes.

5. Electrophoresis of the proteins in polyacrylamide gel (SDS-PAGE) The proteins are separated over an SDS-PAGE electrophoresis gel (sodium dodecyl sulfate polyacrylamide gel, mini-PROTEAN system from Bio-Rad). The concentration of the polyacrylamide gels varies from 8 to 15% as a function of the molecular weight of the proteins to be separated and analyzed. All the separation gels are invariably preceded by a 4% concentration gel according to Table 1 below.

TABLE 1
Composition of the separation and concentration gels.

| | Separation gel (in mL) | | | Concentration |
|---|---|---|---|---|
| For 10 mL | 8% | 12% | 15% | gel (in mL) |
| Distilled water | 5.3 | 4.3 | 3 | 7.25 |
| 40% Acrylamide | 2 | 3 | 3.75 | 1.25 |
| 1.5M Tris (pH: 8.8) | 2.5 | 2.5 | 2.5 | 1.25 |
| 1M Tris (pH: 6.8) | | | | |
| 10% SDS | 0.1 | 0.1 | 0.1 | 0.05 |
| 10% APS | 0.1 | 0.1 | 0.1 | 0.1 |
| TEMED | 0.006 | 0.006 | 0.006 | 0.01 |

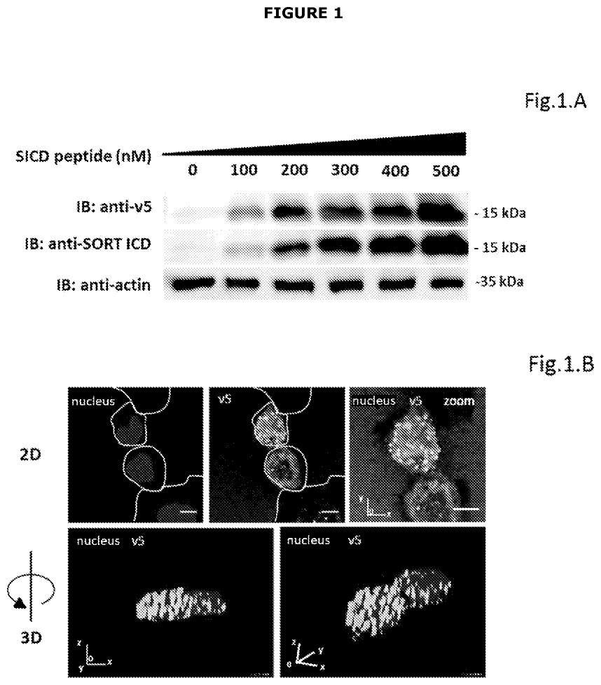

The migration is carried out for 1 hour 30 mins (150 V) in the 1× migration buffer (ref: 1610732, Bio-Rad). A molecular weight marker is used at each migration (ref: 26619 PageRuler™ Plus Prestained Protein Ladder, Fermantas Life Science). Once separated by electrophoresis, the proteins are transferred on a PVDF membrane (polyvinylidene difluoride) (ref: 10600023) Millipore). The membrane is incubated beforehand for 15 seconds in methanol before being rinsed in water then equilibrated for a few minutes in the transfer buffer (ref: 161-0771, Bio-Rad). The polyacrylamide gel is also equilibrated for a few minutes in the transfer buffer before being placed in contact with the membrane. The transfer is carried out for 1 hour at 20 V with a Transblotting ID Dryer appliance (Bio-Rad), according to a setup using Whatman filter papers. The PVDF membrane and the gel are comprised between 2×4 Whatman papers previously soaked with transfer buffer (FIG. 1). The deposit, migration and transfer qualities are verified by a colouration of the PVDF membrane with Ponceau red (0.2% Ponceau S ref: P3504-50 G, Sigma-Aldrich; 3% TCA ref: T8657-250G, Sigma-Aldrich) for a few seconds before being washed 3 times for 5 min with tert-butyl dimethylsilyl (TBS).

6. Incubation of the Membranes with the Antibody

The membranes are then incubated for 1 hour at ambient temperature in TBS 1×containing 0.1% Tween 20 (ref: P1379) and 5% skimmed milk (Regilait), to saturate the nonspecific fixation sites. Immunolabelling is carried out by incubation with the primary antibody, directed against the specific epitopes of the proteins of interest and according to the conditions described in Table 2.

TABLE 2
Primary antibodies used in western blot

| | | | | | | | | Incubation | | | |
|---|---|---|---|---|---|---|---|---|---|---|---|
| | | | | Dilution | | Incubation Time | | Solution | | | PM |
| Antibodies | Clones | Species | Companies | WB¹ | IF² | WB | IF | WB | IF | Ref. | kDa |
| β-actin | — | rabbit | Ozyme | 1/1000 | — | 2-12 h | — | a-b | — | 4970 | 45 |
| Sortilin | | rabbit | Abcam | 1/1000 | 1/200 | 12 h | 12 h | b | b | Ab16640 | 95 |
| Anti-V5 | 2F11F7 | mouse | Invitrogen, | 1/5000 | 1/200 | 12 h | 12 h | b | b | 37- | 5 |
| | | | ThermoFisher | | | | | | | 7500 | |

-: unusable, NA: not reported, (a): TBS 1× Tween 0.1%, Milk 5%, (b): TBS 1×-Tween 0.1%, BSA 3%, (c): TBS 1×, BSA 5%, (d): TBS 1×, BSA 3%, WB: Western Blot, IF: immunofluorescence.

After washing three times in TBS 1×-Tween 20 buffer (0.1%), the membranes are incubated for 1 hour at ambient temperature with the suitable secondary antibody coupled with the peroxidase (Table 3) (dilution at 1/1000 in the saturation solution). The membranes are then washed twice with TBS 1×-Tween 20 (0.1%) then twice with TBS 1× alone to remove the excess Tween 20.

TABLE 3
Secondary antibodies used in western blot HRP: Horseradish Peroxidase

| Secondary antibodies | Host | Supplier | Dilution |
|---|---|---|---|
| Anti-mouse IgG HRP | Rabbit | Dako | 1/1000 |
| Anti-rabbit IgG HRP | Pig | Dako | 1/1000 |

7. Visualization by Chemiluminescence Reaction

Western Blot visualization is carried out by chemiluminescence. This system is based on oxidation of luminol by 02, produced by peroxidase action on H₂O₂. An unstable intermediate light-emitting compound is formed. The membrane is contacted for 1 minute with the mixture of the “ECL” kit (ref: WBULS0500, Millipore) in the proportion 1:1. The chemiluminescence is collected by the G box digital system (Ozyme). The digital images obtained from the western blot visualization are processed using Genesnap (Syngene) and ImageJ (NIH) image analysis software.

8. Indirect Immunofluorescence

The cells are seeded on a 24-well plate on 14 mm diameter glass slides. After each treatment condition, the cells are fixed, either with the complete medium containing paraformaldehyde (PFA) at 3.7% for 20 min at 4° C., or with methanol at −20° C. for 5 min. With methanol the fixation is carried out by dehydration (denaturing of the proteins), while with the aldehydes (formaldehyde) the 3D structure of the protein is preserved (fixing by bridging). The phosphorylations are analyzed after fixing with PFA. After fixing, the cell layer is washed 3 times for 5 minutes with 500 pL PBS. In the case of fixing with PFA, the cells are permeabilized with Triton X-100 at 0.1% in PBS for 10 min at 4° C., then washed again. With regard to fixing with methanol, this confers the advantage of fixing and permeabilizing the cells at the same time. Saturation of the nonspecific sites is carried out with 1% PBS-serum (of the same origin as the animal from which the secondary antibody originates) or with 5% PBS-BSA for 1 hour at ambient temperature. Starting from the fixing stage, all the solutions are filtered beforehand over a 0.2 μm filter in order to eliminate the fluorescence artefacts and the background noise. The primary antibody directed against the protein of interest, (i.e., the V5 tag (allowing visualization of the synthetic peptide)) is incubated overnight at 4° C. in a PBS solution containing 3% BSA (Table 2). Then, three washes of 5 min in PBS are carried out, before incubation of the secondary antibody coupled with an Alexa fluor 488® (green fluorescence) (Table 4) for 1 hour at ambient temperature in the PBS-BSA solution. After three washes of 5 min in PBS, the nuclei are labelled with a PBS 1× solution containing 1 μM fluorochrome DAPI for 5 min. At the end of this stage, the cells are rinsed again with three washes of 5 min in PBS. The slides are then placed on object slides using an aqueous mounting medium (Dako) and placed overnight at 4° C. The preparations are visualized with a confocal microscope of the LSM 880 type (Zeiss) equipped with a helium/neon and argon laser (×63 or ×100 magnification). The images are processed with Zen (Zeiss) or Image J (NIH) software.

TABLE 4
Secondary antibodies used in immunofluorescence

| Secondary antibodies | Host | Supplier | Dilution |
|---|---|---|---|
| Anti-mouse IgG Alexa Fluor ® | Goat | Invitrogen | 1/1000 |
| 488 | | | |

9. Cell Death Analysis with Real-Time Imaging Via the IncuCyteZOOM® System

In order to study cell death by apoptosis, the cells are seeded at a concentration of 2000 cells per well in 100 μL of culture medium, in 96-well plates. 24 hours later, the cell medium is removed, then the cells are rinsed once with sterile PBS 1× before adding the culture medium containing the different treatments, including the peptide according to the invention at 600 nM, and the reagents necessary for analysis of cell death. These latter are: IncuCyte® Caspase-3/7 Green Reagent (ref: 4440; EssenBio) diluted to 1:1000 in the culture medium; IncuCyte® Annexin V Green Reagent (ref: 4642; EssenBio) diluted to 1:200 diluted in the culture medium. Image acquisition is then carried out by positioning the plates in the IncuCyte Zoom® system (Ref: EssenBio) for 96 hours. Four images per well are then obtained, in high definition, every two hours, with the ×10 objective and the phase-contrast and green channels. These acquisitions are carried out automatically and non-invasively. The data are processed automatically with the IncuCyte Zoom® Live-Cell Analysis System (ref: EssenBio).

### Example 1: Cell Penetration Test Via Western Blot and Immunofluorescence

This is conducted according to the technique described above. The synthetic peptide of sequence SEQ ID NO 2 (SICD or sortilin intracellular domain) is rendered competent to penetrate the cells by addition of a Tat sequence (trans-activator of transcription), SEQ ID NO 8 belonging to the family of cell-penetrating peptides of the human immunodeficiency virus (HIV). Furthermore, a V5 tag (SEQ ID NO 10), epitope known for easy detection via a specific antibody or a fluorescence molecule, is grafted on the peptide, then leading to the synthetic peptide of sequence SEQ ID NO 7. The cells are treated with a concentration range of the SICD peptide coupled with the V5 tag and the Tat of sequence SEQ ID NO 7, ranging from 0 to 500 nM diluted in complete culture medium. After 24 hours' treatment, the cells are lysed and the peptide is detected in the cell lysate (intracellular medium) with an anti-V5 antibody. Following the different stages of western blot, a progressive increase in the quantity of intracellular peptide is detected with a maximum at the highest concentration (500 nM) thus suggesting a dose-dependent peptide penetration, as illustrated in FIG. 1.A line IB: anti-V5. Actin acts as internal control of deposit, demonstrating that an identical quantity of protein has been deposited in each protein track presented (FIG. 1.A line IB: anti-actin). A similar profile is obtained by using an antibody directed against the intracellular portion of sortilin (FIG. 1.A line IB: anti-SORT ICD). These results confirm on the one hand that the peptide is in fact internalized by the cells, and on the other hand that this is indeed a peptide derived from the intracellular portion of sortilin.

The subcellular distribution of the synthesized peptide was visualized by immunofluorescence (FIG. 1.B). The images show a punctiform nuclear distribution of the synthetic peptide of sequence SEQ ID NO 7 by using an antibody directed against the V5 tag. This distribution is also confirmed by using the peptide of sequence SEQ ID NO 7 and an anti-sortilin antibody. The superposition of the labels (DAPI labelling the cell nuclei and V5 labelling the synthesized peptide) confirms the specific presence of the peptide in the cell nuclei. The images taken in 3D confirm this location within the nuclei, and not on the surface thereof. The presence of the synthetic peptide in this cellular compartment is evidence of the action thereof at the level of the nucleus.

### Example 2: Measurement of the Antineoplastic Effect by Measuring Cell Survival

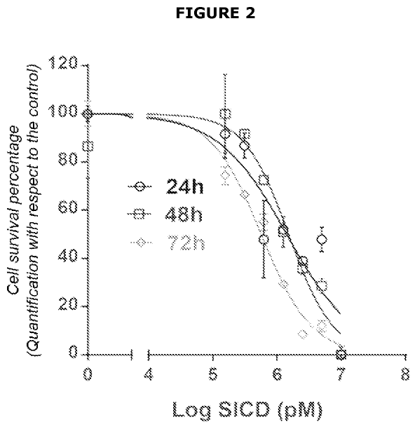

The antineoplastic effect of the synthetic peptide, comprising Tat and a V5 tag of sequence SEQ ID NO 7 was tested on the A549 cell line (adenocarcinoma line having no mutation on the EDFR). A concentration range (0 to 10 μM) of the synthetic peptide of sequence SEQ ID NO 7 according to the invention was incubated for 24, 48 and 72 hours in the cell medium and the dehydrogenase activity thereof (proxy for viability) was measured (Cell Proliferation Kit II (XTT)-Roche) at the end of each incubation, i.e. at 24, 48 and 72 hours. The results are given in FIG. 2. Analysis of the graph representing the activity as a function of the log of the synthetic peptide concentration made it possible to establish CI₅₀ values corresponding to the concentrations for which the peptide results in 50% inhibition of cell activity. These latter are respectively equal to 1.42, 1.52 and 0.57 μM for incubation times of 24, 48 and 72 hours.

### Example 3: Measurement of the Pro-Apoptotic Effect of the Synthetic Peptide

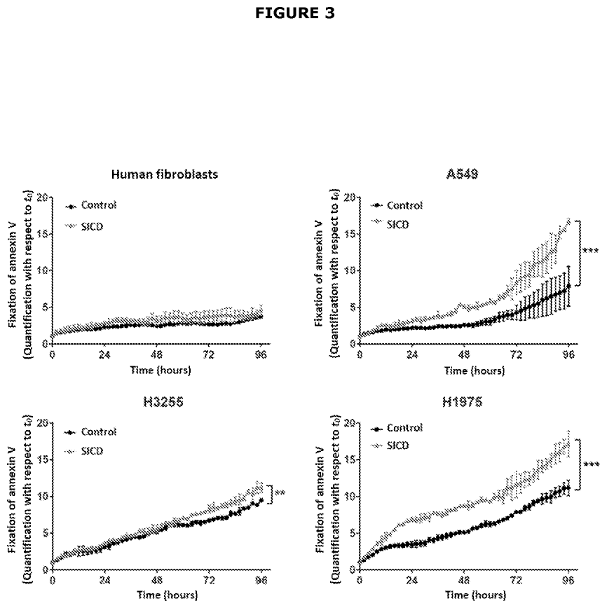

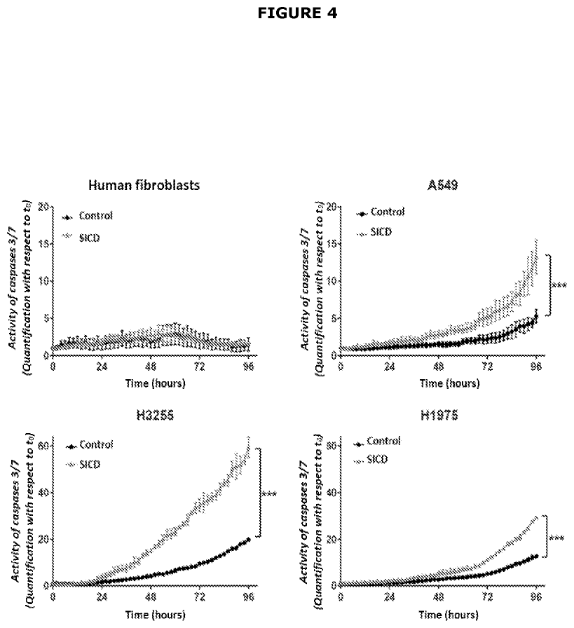

The pro-apoptotic effect of the peptide of sequence SEQ ID NO 7 at the concentration of 1 μM was measured in real time using the IncuCyte Zoom® system for 96 hours on the A549 line, on human fibroblasts (non-cancerous cells), as well as on two lines of adenocarcinomas H3255 and H1975, respectively presenting mutations of EGFR that are sensitive (L858R) and resistant (L858R/T790M) to the tyrosine kinase activity receptor inhibitors (TKI) used in the clinic via activation of two markers of apoptosis, namely annexin V and caspases 3 and 7. The results show that the peptide does not seem to lead to activation of these two markers of apoptosis in the human fibroblast culture (annexin V FIG. 3 and caspases 3 and 7 FIG. 4). In fact, the levels of expression of these markers are identical under the conditions treated with the peptide and the control conditions. The peptide could therefore have a specific toxicity to cancer cells.

Conversely, on the three adenocarcinoma lines tested, an increase is observed over time in the apoptotic process in the cells. In fact, the levels of expression of annexin V (FIG. 3) or of caspases 3 and 7 (FIG. 4) increase in the three adenocarcinoma lines tested, reflecting an action mechanism of the peptide with respect to the antineoplastic effect thereof.

Unlike the molecules available in conventional chemotherapy, the synthetic peptide according to the invention results from an amino acid assembly to form a peptide that is naturally present in human cells and not from chemical molecules. Therefore, improved tolerance by the organism is envisaged. Owing to the ease of synthesis thereof, at reasonable cost, and its specific intracellular target, different from those of the TKIs, the peptide according to the invention could be a good candidate for use in bitherapy. In this case, it would allow a reduction in the doses of conventional antineoplastics administered, and a reduction in the associated treatment costs.

These results show that after administration thereof, the peptide according to the invention is in fact present inside the nuclei (FIG. 1) and that it is capable of inhibiting the activity of tumour cells (FIG. 2) and has a pro-apoptotic effect (FIG. 3). Therefore, this peptide can be used in diseases associated with sortilin deregulation, in particular in the case where sortilin is suppressed

1. An isolated polypeptide derived from human sortilin comprising: an amino acid sequence having at least 80% identity with sequence SEQ ID NO 2
Val Leu Ile Val Lys Lys Tyr Val Cys Gly Gly Arg Phe Leu Val His Arg Tyr Ser Val Leu Gln Gln His Ala Glu Ala Asn Gly Val Asp Gly Val Asp Ala Leu Asp Thr Ala Ser His Thr Asn Lys Ser Gly Tyr His Asp Asp Ser Asp Glu Asp Leu Leu Glu
provided that said polypeptide does not contain sequence SEQ ID NO 3 or a sequence having at least 80% identity with said sequence SEQ ID NO 3 or one of its pharmaceutically acceptable salts for use thereof as a medicament.

2. The polypeptide for use thereof as a medicament according to claim 1, characterized in that said peptide is covalently bound to a cell-penetrating peptide.

3. The polypeptide for use thereof as a medicament according to claim 2, characterized in that said cell-penetrating peptide is selected from the group comprising HIV-1 Tat, penetratin, peptide originating from the homeodomain of the Antennapedia protein, p28 originating from azurin, VP22 from the herpes simplex virus (HSV-1), the synthetic MAP peptide of sequence SEQ ID NO 11 or DPV1047 of sequence SEQ ID NO 12.

4. The polypeptide for use thereof as a medicament according to claim 1 in the treatment of a pathology associated with sortilin deregulation, selected from the group comprising cancers, in particular non-small cell lung cancers, neurological disorders, in particular Parkinson's disease, Alzheimer's disease, coronary diseases and atherosclerosis.

5. The polypeptide for use thereof as a medicament according to claim 1, characterized in that it is presented in a pharmaceutical form suitable for local, regional, systemic or continuous administration.

6. The polypeptide for use thereof as a medicament according to claim 1 in association with at least one second molecule, selected from antineoplastic chemotherapeutic molecules, tyrosine kinase receptor inhibitors, signalling pathway inhibitors used in targeted treatments, antibodies specific for oncogene receptors, T-lymphocyte checkpoint inhibitors, hormones, toxins and radiotherapy agents.

7. An isolated polypeptide derived from human sortilin, comprising; an amino acid sequence having at least 80% identity with sequence SEQ ID NO 2 Val Leu Ile Val Lys Lys Tyr Val Cys Gly Gly Arg Phe Leu Val His Arg Tyr Ser Val Leu Gln Gln His Ala Glu Ala Asn Gly Val Asp Gly Val Asp Ala Leu Asp Thr Ala Ser His Thr Asn Lys Ser Gly Tyr His Asp Asp Ser Asp Glu Asp Leu Leu Glu
provided that said polypeptide does not contain sequence SEQ ID NO 3 or a sequence having at least 80% identity with said sequence SEQ ID NO 3; and
said peptide is covalently bound to a cell-penetrating peptide.

8. The polypeptide according to claim 7, characterized in that said cell-penetrating peptide is selected from the group comprising HIV-1 Tat, penetratin, peptide originating from the homeodomain of the Antennapedia protein, p28 originating from azurin, VP22 from the herpes simplex virus (HSV-1) or the synthetic peptide MAP of sequence SEQ ID NO 11 or DV.

9. An isolated nucleotide sequence selected from the following polynucleotides:
a. a polynucleotide coding for the polypeptide according to claim 7 of sequence SEQ ID NO 4 and consisting of sequence SEQ ID NO 6,
b. a polynucleotide that has at least 80%, at least 85%, at least 90%, at least 95% homology with the polynucleotide of sequence SEQ ID NO 6 and
c. a polynucleotide that is degenerate with respect to polynucleotides a and b.

10. An expression vector comprising a polynucleotide according to claim 9.

11. A host cell comprising a polynucleotide coding for a polypeptide according to claim 9.

12. A pharmaceutical composition comprising a polypeptide according to claim 7 or a pharmaceutically acceptable salt thereof, in combination with at least one pharmaceutically acceptable excipient.

13. The composition according to claim 12, also comprising another active agent selected from chemotherapeutic molecules, tyrosine kinase receptor inhibitors, signalling pathway inhibitors used in targeted treatments, antibodies specific for oncogene receptors, T-lymphocyte checkpoint inhibitors, hormones, toxins and radiotherapy agents.

14. An isolated polypeptide derived from human sortilin, comprising; an amino acid sequence having at least 80% identity with sequence SEQ ID NO 2
Val Leu Ile Val Lys Lys Tyr Val Cys Gly Gly Arg Phe Leu Val His Arg Tyr Ser Val Leu Gln Gln His Ala Glu Ala Asn Gly Val Asp Gly Val Asp Ala Leu Asp Thr Ala Ser His Thr Asn Lys Ser Gly Tyr His Asp Asp Ser Asp Glu Asp Leu Leu Glu
provided that said polypeptide does not contain sequence SEQ ID NO 3 or a sequence having at least 80% identity with said sequence SEQ ID NO 3
and pharmaceutically acceptable salts thereof,
said peptide being covalently bound or not to a cell-penetrating peptide for use thereof as a marker of pathologies associated with sortilin deregulation, in particular cancers, in particular non-small cell lung cancers, neurological disorders, in particular Parkinson's disease and Alzheimer's disease, coronary diseases and atherosclerosis.

15. A pharmaceutical composition comprising a polynucleotide according to claim 9 or a pharmaceutically acceptable salt thereof, in combination with at least one pharmaceutically acceptable excipient.
